(19) United States
(12) Patent Application Publication
(10) Pub. No.: US 2026/0262918 A1
(43) Pub. Date: Sep. 10, 2026

(54) IMAGING SYSTEM FOR MEDICAL ENDOSCOPIC SYSTEM, WHICH REMOVES HONEYCOMB ARTIFACTS FROM IMAGES FROM MULTICORE OPTICAL FIBRES

(71) Applicant: AXESS VISION TECHNOLOGY, JOUE LES TOURS (FR)

(72) Inventors: Xavier DIEUDONNÉ, MONTS (FR); Emmanuel HALLAUER, SACHE (FR)

(73) Assignee: AXESS VISION TECHNOLOGY, JOUE LES TOURS (FR)

(21) Appl. No.: 19/157,951

(22) PCT Filed: Dec. 8, 2023
(86) PCT No.: PCT/FR2023/051962

§ 371 Date: Aug. 19, 2025

(30) Foreign Application Priority Data
Dec. 8, 2022 (FR) FR2212974

(51) Int. Cl. A61B 1/00; A61B 1/06; A61B 1/07

(52) U.S. Cl. CPC A61B 1/000095; A61B 1/0669; A61B 1/07

(57) ABSTRACT

An imaging system for a target including a medical endoscopic system provided with two multicore optical fibers each having a plurality of cores separated by a separating structure. The imaging system further includes a device for acquiring and processing images. The device includes at least one illumination source configured and at least one imaging sensor configured to receive light beams coming from the multicore optical fibers, the imaging sensor receiving the images from a configuration system to create, on the imaging sensor, images of the target, for which the position of the separating structure of the first multicore optical fiber is different from the position of the separating structure of the second multicore optical fiber. The device further includes an imaging processor for reconstituting a composite image of the target.

### TECHNICAL FIELD

This invention relates to the technical field of imaging systems implemented in the context of medical endoscopic systems in the general sense, used to access the inside of a body such as a cavity or a canal for example, and more specifically pertains to endoscopic medical systems: medical catheters and medical endoscopes.

The medical endoscopic system implemented in the context of this invention has particularly advantageous applications in making it possible to access the inner surface of a hollow organ, a cavity or a natural or artificial duct of the human body for the purpose of carrying out various operations for therapeutic, surgical or diagnostic purposes, and which can be used in the field of urinary tracts, gastro-intestinal tracts, the respiratory system, the cardiovascular system, the trachea, the sinus cavity, the female reproductive system, the abdominal cavity or any other part of the human body to be explored via a natural or artificial pathway.

### PRIOR ART

Conventionally, a medical endoscopic system of medical catheter or medical endoscope type includes a control handle to which is attached an insertion tube having, opposite its part attached to the control handle, a distal head. This insertion tube has a greater or lesser length and flexibility so as to be able to be introduced into a natural or artificial pathway for the purpose of carrying out various operations or functions for therapeutic, surgical or diagnostic purposes. Note that such an endoscopic system is designed to have the smallest possible section to be able to access pathways of limited through section.

For a medical endoscopic system of endoscope type, the distal head is equipped in particular with a viewing system making it possible to examine the organ, cavity or duct of the human body. Upstream of this distal head, the insertion tube includes a flexible or articulating part formed of articulated vertebra allowing the distal head to be pivoted. This medical endoscope is intended to be connected to a medical electronic device including a unit for processing the image signals delivered by the viewing system of the endoscope. The images taken are viewed on a screen of this device or on a remote screen connected to this device.

The viewing system mounted on the distal part of the tube includes a camera associated or not associated with one or more light sources such as light-emitting diodes. The camera, or even the light sources are electrically connected to electrical components located in the handle or in the medical device. According to the exemplary embodiment described by patent application US 2022/0160218, the camera and light sources located at the distal part of the insertion tube are connected to the electrical components located in the handle. Note that a medical endoscopic system is generally used in an environment in which various electrical equipment items are in operation such as electrosurgical scalpels, X-ray devices, scanners or screens, liable to affect the operation of the camera and/or the signal delivered by the camera. Moreover, in the case of a disposable endoscopic system, the light sources and the camera are disposed of as waste. In addition, these electronic components are electronic waste requiring recycling, increasing the cost of such a system.

There is also known from patent US 11 061 185 a medical endoscopic system including a multicore optical fiber composed of a large number of cores separated by a matrix and housed in a common sheath. This multicore fiber receives the radiation from a target at its distal end and transmits the radiation over its entire length to deliver the radiation, at its proximal part, to an imaging sensor. The medical endoscopic system also includes an illumination source delivering, at the proximal end of an illuminating optical fiber, a light beam departing from the distal end of the illuminating fiber to illuminate the target.

Such a medical endoscopic system requires the use of a multicore optical fiber and of an illuminating optical fiber for the purpose of obtaining a quality image of the target. However, given the implementation of multicore fibers, a honeycomb-shaped outline, corresponding to the matrix separating the cores of these multicore optical fibers, appears on the images taken. This separating structure hinders the observation of the part of the target located in a matching relationship with this separating structure.

### SUMMARY OF THE INVENTION

The subject of the invention has the aim of remedying the drawbacks of the prior art by making provision for an imaging system making it possible to obtain a complete image of the target while having an insensitivity to electromagnetic disturbances and a reduction of the electronic components that end up as waste.

To achieve this objective, the imaging system according to the invention for a medical endoscopic system for viewing a target includes:
a medical endoscopic system including an insertion instrument terminating, opposite a proximal part, in a distal head, this insertion instrument being provided with a first multicore optical fiber and with a second first multicore optical fiber each having a distal end, a proximal end and a plurality of cores, separated by a separating structure, the distal ends of the first multicore optical fiber and of the second multicore optical fiber being located at the distal head of the insertion instrument;
a device for acquiring and processing images including:
at least one illumination source configured to deliver a light beam, in at least a first wavelength spectrum, to the distal head of the insertion instrument;
at least one imaging sensor, configured to receive light beams coming from at least the proximal end of the first multicore optical fiber and from the proximal end of the second multicore optical fiber, the imaging sensor receiving the images from a configuration system to create, on the imaging sensor, images of the target, for which the position of the honeycomb of the first multicore optical fiber is different from the position of the honeycomb of the second multicore optical fiber;
an imaging processor connected to the imaging sensor and configured to process the images coming from the first multicore optical fiber and from the second multicore optical fiber, to reconstitute a composite image of the target in which the image of the honeycomb does not appear.

According to an embodiment, the device for acquiring and processing images includes a single imaging sensor configured to either receive, on two separate areas, the light beams coming from the first multicore optical fiber and from the second multicore optical fiber, or to receive, on a shared but time-offset area, the light beams coming from the first multicore optical fiber and from the second multicore optical fiber.

According to another embodiment, the device for acquiring and processing images includes two imaging sensors, configured to each receive a light beam coming either from the first multicore optical fiber or from the second multicore optical fiber.

According to an advantageous feature of implementation, the configuration system is configured so that the images coming from the first multicore optical fiber and the images coming from the second multicore optical fiber can be matched.

Advantageously, the imaging processor processes the images to determine an orientation of the distal head.

According to an exemplary embodiment, the configuration system is embodied by the first multicore optical fiber and the second multicore optical fiber having sections of different shapes.

According to another exemplary embodiment, the configuration system is embodied by the first multicore optical fiber and the second multicore optical fiber having different orientations.

According to another exemplary embodiment, the configuration system is embodied by a first multicore optical fiber and a second multicore optical fiber having different numerical apertures.

According to another exemplary embodiment, the configuration system is embodied by an optical system disposed at the distal end of the multicore optical fibers to create different field depths.

According to another exemplary embodiment, the configuration system is embodied by the first multicore optical fiber having cores of a given diameter while the second multicore optical fiber has cores with a different diameter from the diameter of the cores of the first optical fiber.

According to another exemplary embodiment, the configuration system is embodied by the first multicore optical fiber having a section of given shape while the second multicore optical fiber has a section of different shape from the shape of the section of the first multicore optical fiber.

Advantageously, the illumination source is configured to deliver a light beam to at least one multicore optical fiber.

According to another exemplary implementation, an illumination source is configured to deliver a light beam to an optical fiber conveying the light all the way to the distal head of the insertion instrument.

According to an advantageous feature of implementation, the imaging sensor or sensors receive the images from the configuration system configured to create, on the imaging sensor or sensors, images coming from the two multicore optical fibers and containing size indicators of the target and the imaging processor processes the images to determine a measurement of the target based on the size indicators.

### BRIEF DESCRIPTION OF THE DRAWINGS

FIG. 1 is an overall view of an example of an application of a remote imaging system for an endoscope as the medical endoscopic system for viewing a target.

FIG. 2 is an overall view of another example of an application of a remote imaging system for an endoscope as the medical endoscopic system for viewing a target.

FIG. 3 is an overall view of another example of an application of a remote imaging system for a catheter as the medical endoscopic system for viewing a target.

FIG. 4 shows an example of a super-resolved image in which the honeycomb present in two images obtained using two multicore optical fibers no longer appears.

FIG. 5 schematically represents another exemplary embodiment of a remote imaging system for a medical endoscopic system implementing two multicore optical fibers, one light source and one imaging sensor.

FIG. 6 schematically represents another exemplary embodiment of a remote imaging system for a medical endoscopic system implementing two multicore optical fibers, two light sources and one imaging sensor.

FIG. 7 schematically represents another exemplary embodiment of a remote imaging system for a medical endoscopic system implementing two multicore optical fibers, one light source and two imaging sensors.

FIG. 8 schematically represents another exemplary embodiment of a remote imaging system for a medical endoscopic system implementing two multicore optical fibers, two light sources and two imaging sensors.

FIG. 9 schematically represents the distal head of a medical endoscope including two multicore optical fibers and showing a detail of a multicore optical fiber.

FIG. 10 is a functional block diagram of an exemplary embodiment of a remote imaging system for a medical endoscopic system.

FIG. 11 schematically represents another exemplary embodiment of a remote imaging system for a medical endoscopic system implementing two multicore optical fibers, one light source and two imaging sensors as well as an additional illuminating fiber.

FIG. 12 schematically represents the distal head of a medical endoscope including two multicore optical fibers, for which the positions of the matrices are different, making it possible to obtain images with offset matrix positions for the target.

FIG. 13 schematically represents the distal head of a medical endoscope including several illuminating fibers and two multicore optical fibers, the cores of which have cores of different diameters.

FIG. 14 schematically represents the distal head of a medical endoscope including two multicore optical fibers with sections of different shapes.

FIG. 15 schematically represents another exemplary embodiment of an imaging system including an optical system disposed at the distal end of two multicore optical fibers to create different field depths.

### DESCRIPTION OF THE EMBODIMENTS

As can be seen from the figures, the subject of the invention relates to an imaging system I for a medical endoscopic system 1 of endoscope or catheter type in the general sense designed to access the inside of a body such as a cavity or a canal for example. Conventionally, an endoscopic system 1 of endoscope or catheter type includes an insertion instrument 2 having a proximal part 2a and opposite it, a distal part forming a free end. The insertion instrument 2 thus terminates at its free end in a distal head 2b from which is viewed a target C in the general sense.

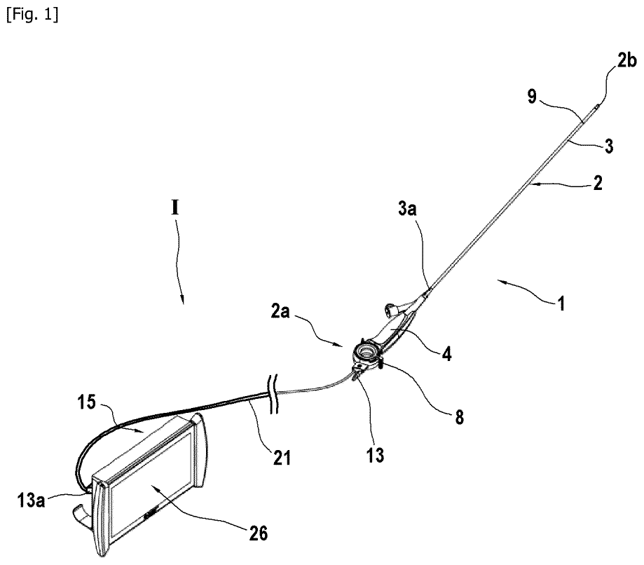

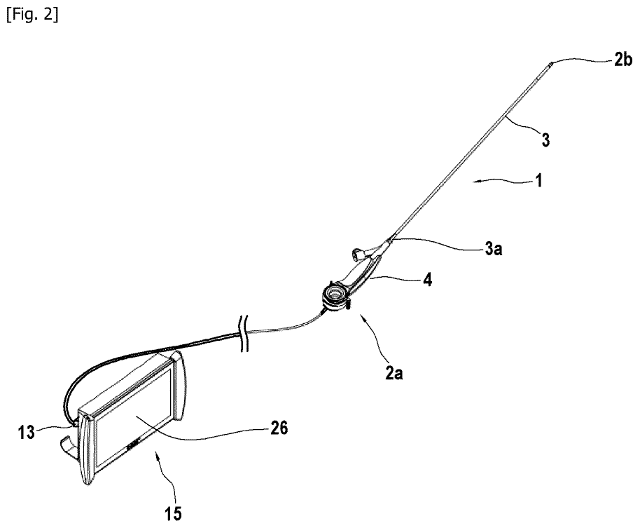

According to a mode of application for which the medical endoscopic system 1 is an endoscope (FIGS. 1, 2 and 9), the medical endoscopic system includes as the insertion instrument 2 an insertion tube 3 having a free end forming the distal head 2b and held at its opposite end by a control handle 4, all or part of which forms the proximal part 2a of the insertion instrument. The insertion tube 3 is temporarily or permanently attached to the control handle 4. In the example illustrated in FIGS. 1 and 2, the insertion tube 3 is engaged by its opposite end with its free end, in a housing of an endpiece 3a intended to be attached to the distal part of the control handle 4. This insertion tube 3, which has a greater or lesser length and flexibility, is intended to be introduced into a natural or artificial access pathway for the purpose of performing various operations or functions for therapeutic, surgical or diagnostic purposes.

The insertion tube 3 is made of a semi-rigid material such as for example thermoplastic elastomer (TPE). The insertion tube 3 has a length suitable for the length of the duct to be inspected and able to be between 5 cm and 3 m. The insertion tube 3 has various shapes of cross section such as square, oval or circular. This insertion tube 3 which is in contact with the tissues, human organs or medical appliances (trochars or probes), essentially lies within the category of single or multiple use on one patient or even of re-usable use after decontamination, disinfection or sterilization.

The endoscopic system 1 of endoscope type also includes inside the insertion tube 3 a tubular duct 6 forming an operating or work channel extending from the control handle 4 all the way to the distal head 2b to allow at this distal head, the entry of various tools and/or delivery of fluids and/or suction of fluids (FIG. 9). The tubular duct 6 is surrounded by the insertion tube 3 over its entire length between the distal head 2b and the control handle 4. Conventionally, the tubular duct 6 extends beyond the endpiece 3a inside the control handle 4.

Conventionally, the endoscopic system 1 of endoscope type also includes a control mechanism 8 making it possible to orient the distal head 2b with respect to the longitudinal axis of the insertion tube 3. For this purpose, the insertion tube 3 includes, upstream of the distal head 2b, a flexible, bending or articulating structure 9 allowing the orientation of the distal head 2b with respect to the longitudinal axis of the insertion tube 3. The control mechanism 8 can be embodied in any appropriate manner such that the distal head 2b can be displaced between an idle position in which the insertion tube 3 is rectilinear and articulated position in which the articulating part 9 is curved. For example, the control mechanism 8 may include a manual control lever rotationally driving a pulley to which is attached at least one actuating cable mounted to be attached at the distal head 2b.

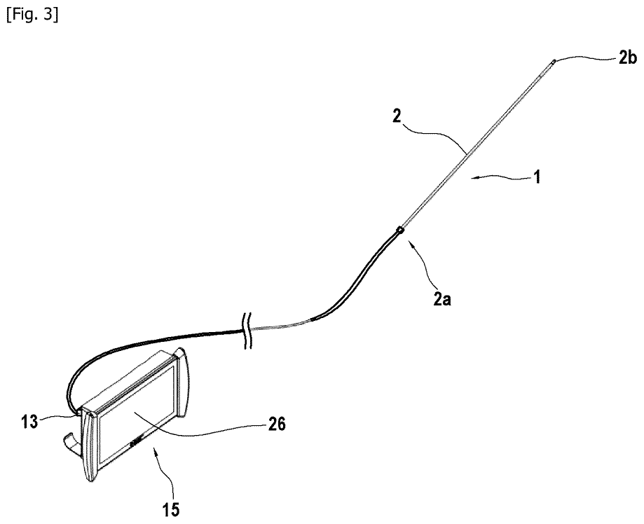

According to another mode of application for which the medical endoscopic system 1 is a catheter (FIG. 3), the medical endoscopic system includes as the insertion instrument 2 a catheter terminating opposite a proximal part 2a in a distal head 2b. Said catheter may be of conventional design with no actuating system making it possible to move the distal part along one or more axes. It can also be equipped with a deflection system with return to position by a shape memory structure, such as for example a plate or a thread made of nitinol.

Another actuating device of the distal part can be embodied by means of cables, of deformable parts by making use of the elasticity of the materials.

In accordance with the invention, the insertion instrument 2 is provided with at least a first multicore optical fiber 11 and a second multicore optical fiber 12. Each multicore optical fiber 11, 12 respectively has a distal end 11a, 12a and a proximal end 11b, 12b. The distal end 11a, 12a of the multicore optical fibers is located at the distal head 2b of the insertion instrument 2 in such a way as to view the target C. Each multicore optical fiber 11, 12 extends at least all the way to the proximal part 2a of the insertion instrument and is provided with at least one optical connector 13 at its proximal end 11b, 12b.

Of course, the optical connector 13 equipping the proximal end 11b, 12b of the multicore optical fibers 11, 12 is intended to interact with a complementary male or female optical connector according to the female or male type of the optical connector 13. Advantageously, a focusing lens is mounted in the complementary connector making it possible to improve the optical connection, offering a wider positioning tolerance. Specifically, the optical connector 13 may be intended to be disposed of as waste with the insertion instrument. In this case, the optical connector 13 can be embodied economically with significant allowances for tolerance.

In a known manner, a multicore optical fiber 11, 12 is an optical fiber including a large number of cores 11 c, 12 c (FIG. 9), for example at least 10 000 cores separated by a common cladding or a separating structure such as a matrix 11d, 12d. These cores 11c, 12c clad in the separating structure 11d, 12d are mounted inside a shared protective sheath 11e, 12e. This structure 11d, 12d for separating the cores 11c, 12c from one another has, along the section of the multicore optical fiber, a honeycomb shape which appears on the images taken, in the form of a dark area G as illustrated on FIG. 4. For example, the following may be used as multicore optical fibers 11, 12: optical fibers marketed under the trade name ESKA by the Mitsubishi Rayon Co. company, MBI by the Asahi Kasei company or else FIGP by the Fujikura company.

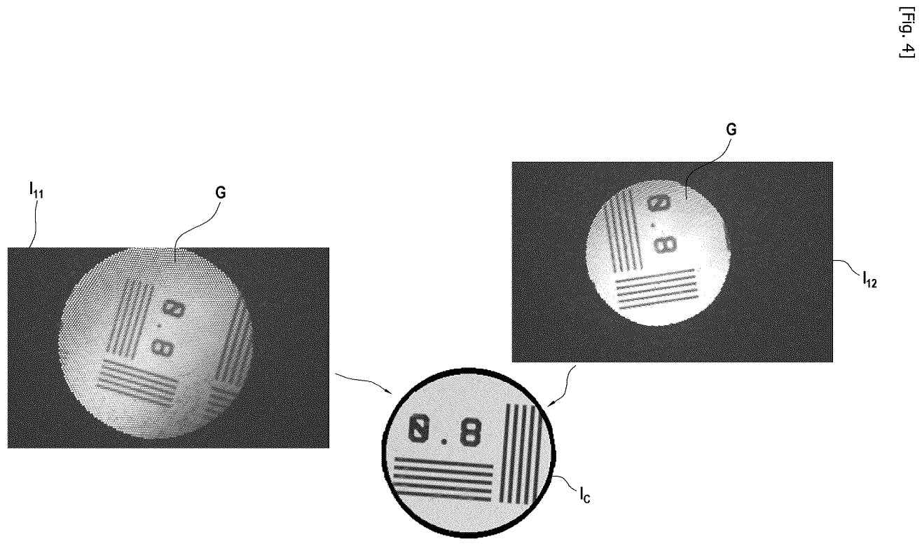

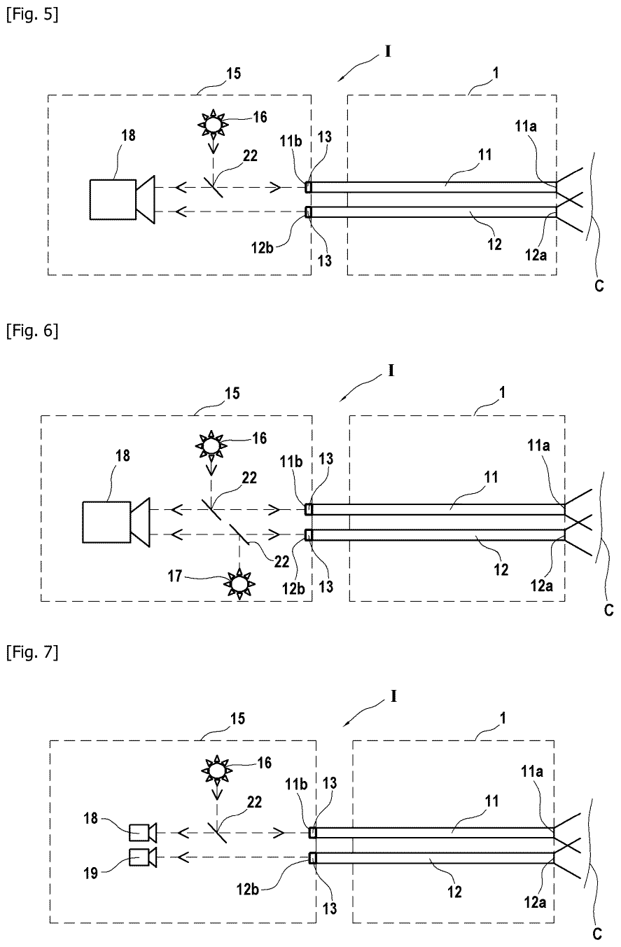

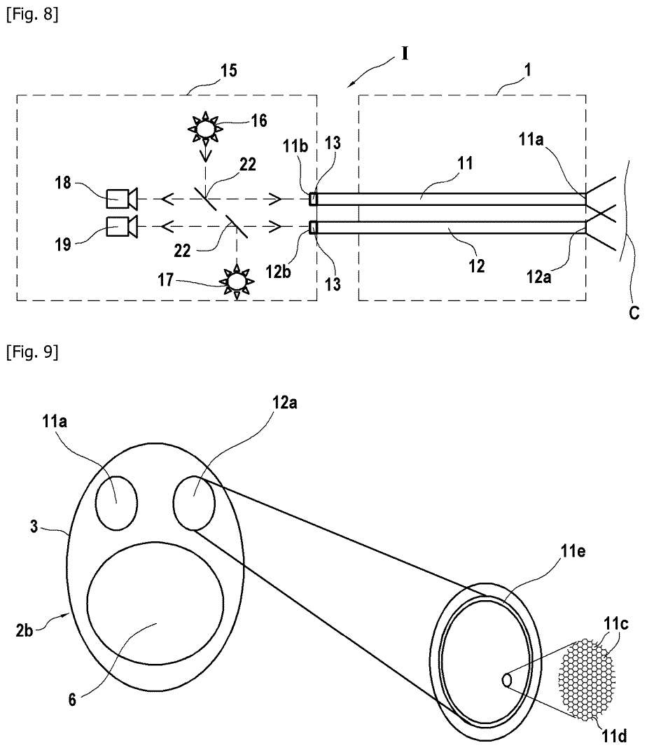

The imaging system I also includes a device 15 for acquiring and processing images including a single illumination source 16 as in the variants illustrated in FIGS. 5 and 7 and a first illumination source 16 and a second illumination source 17 as in the variants illustrated in FIGS. 6 and 8. Each illumination source 16, 17 is configured to deliver a light beam in at least a first wavelength spectrum, to a multicore optical fiber 11, 12 via the optical connector 13. Each illumination source 16, 17 is embodied in any appropriate way to allow the multicore optical fiber 11, 12, to deliver to its distal end 11a, 12a a light beam suitable for illuminating the target C to be imaged. For example, the illumination sources 16, 17 can be embodied by light-emitting diodes, halogen lamps, or infrared or ultraviolet radiation light sources. The device 15 for acquiring and processing images also includes at least one imaging sensor 18 as in the variant embodiments illustrated in FIGS. 5 and 6 and a first imaging sensor 18 and a second imaging sensor 19 as in the variant embodiments illustrated in FIGS. 7 and 8. Each imaging sensor 18, 19 is configured to receive a light beam coming from the proximal end of a multicore optical fiber 11, 12, each equipped with the optical connector 13.

As can be seen from the different variant embodiments, it should be noted that the illumination sources 16, 17 and the imaging sensors 18, 19 are part of the device 15 for acquiring and processing images and are thus remote from the medical endoscopic system 1. It ensues that in the case where the medical endoscopic system 1 is of disposable type, the illumination sources 16, 17 and the imaging sensors 18, 19 can be reused with another medical endoscopic system 1, thus reducing the electronic waste. Moreover, in the scenario where the medical endoscopic system 1 requires a decontamination operation, the device 15 for acquiring and processing images is not concerned by such an operation so that the illumination sources 16, 17 and the imaging sensors 18, 19 which are part of this device are not liable to be damaged by this decontamination operation.

Note that in the example of an application illustrated in FIG. 1 for which an endoscope is used as the medical endoscopic system 1, the proximal end of the multicore optical fiber or fibers, provided with the optical connector 13, is located at the proximal part 2a of the insertion instrument i.e. at the control handle 4. The multicore optical fibers 11, 12 are mounted inside the insertion tube 3 but outside the tubular duct 6. The multicore optical fibers 11, 12 thus extend from the distal head of the insertion tube, being inserted over the entire length of the insertion tube 3, between this latter and the tubular duct 6. The multicore optical fibers 11, 12 extend inside the control handle 4 all the way to one or more optical connectors 13 mounted at the proximal part of the control handle 4.

According to this example, an optical cable 21 provides an optical link between the optical connector or connectors 13 and the device 15 for acquiring and processing images to convey the light beams between on the one hand, the multicore optical fibers 11, 12 and on the other hand, the illumination sources 16, 17 and the imaging sensor or sensors 18, 19. The optical cable 21 can be embodied in any appropriate way in the form of one or more optical fibers.

Typically, the optical cable 21 is provided opposite its part connected to the optical connector 13, with an optical connector 13a attached to the device 15 for acquiring and processing images.

Note that in the example of an application illustrated in FIG. 2 for which an endoscope is used as the medical endoscopic system 1, the proximal end of the multicore optical fibers, provided with the optical connector 13 is linked directly to the device 15 for acquiring and processing images. According to this example, the proximal end of the multicore optical fibers, provided with the optical connector 13, is attached directly to the device 15 for acquiring and processing images. Thus, the multicore optical fibers 11, 12 extend from the distal head of the insertion tube, being inserted over the entire length of the insertion tube 3, between this latter and the tubular duct 6. The multicore optical fibers 11, 12 extend inside the control handle 4 to come out of the proximal part of the control handle in such a way as to be connected to the optical connector 13 attached to the device 15 for acquiring and processing images.

Note that in the example of an application illustrated in FIG. 3 for which a catheter is used as the medical endoscopic system 1, this catheter is formed at least by the multicore optical fibers 11, 12, the distal end 11a, 12a of which forms the distal head 2b of the insertion instrument 2. If two multicore optical fibers 11, 12 are used, the insertion instrument 2 is formed by a protective sleeve in which the two multicore optical fibers 11, 12 are mounted. Note that the proximal end of the multicore optical fibers 11, 12 is linked via the optical connector 13, directly to the device 15 for acquiring and processing images (as illustrated on FIG. 3) or indirectly using the optical cable 21 (as explained in relation to FIG. 1).

According to a first exemplary embodiment illustrated on FIG. 5, the endoscopic system 1 includes a first multicore optical fiber 11 and a second multicore optical fiber 12. The device 15 for acquiring and processing images includes:
an illumination source 16 configured to deliver a light beam to the first multicore optical fiber 11,
a single imaging sensor 16 configured to have a first area for receiving a light beam coming from the proximal end of the first multicore optical fiber 11 and a second receiving area separate from the first receiving area, for receiving a light beam coming from the proximal end of the second multicore optical fiber 12. Note that the single imaging sensor 16 is configured to either receive, on two separate areas, the light beams coming from the first multicore optical fiber 11 and from the second multicore optical fiber 12, or to receive, on a shared but time-offset area, the light beams coming from the first multicore optical fiber 11 and from the second multicore optical fiber 12.

The device 15 for acquiring and processing images includes an optical separating system 22 disposed on the optical path between the proximal end of the multicore optical fiber 11 and the imaging sensor 18 and reflecting, in the direction of the proximal end of the multicore optical fiber 11, the light beam coming from the illumination source 16. This optical separating system 22 can be embodied by any appropriate means such as by a half-wave plate, a beam splitter or an optical prism system.

This example has the advantage of being able to obtain two images simultaneously which may be processed at the same time to make a super-resolution as will be described in the remainder of the description.

According to a second exemplary embodiment illustrated in FIG. 6, the endoscopic system 1 includes a first multicore optical fiber 11 and a second multicore optical fiber 12. The device 15 for acquiring and processing images includes:
a first illumination source 16 configured to deliver a light beam to the first multicore optical fiber 11,
a second illumination source 17 configured to deliver a light beam to the second multicore optical fiber 12,
a single imaging sensor 18 configured to have a first area for receiving a light beam coming from the proximal end of the first multicore optical fiber 11 and a second receiving area separate from the first receiving area, for receiving a light beam coming from the proximal end of the second multicore optical fiber 12.

The device 15 for acquiring and processing images includes an optical separating system 22 disposed on the optical path between the proximal end of each multicore optical fiber 11, 12 and the imaging sensor 18 and reflecting, in the direction of the proximal end of each multicore optical fiber 11, 12, the light beam coming from the illumination source 16.

This exemplary embodiment makes it possible to illuminate the target with light beams having different wavelength spectra in order to obtain a spectral super-resolution image. This solution offers the advantage of being able to view tumors. Specifically, by choosing a specific wavelength spectrum, the vascularization of the tissues can be highlighted. As a tumor is a highly vascularized area, a tumor can be more closely observed by implementing this technique.

According to a third exemplary embodiment illustrated on FIG. 7, the endoscopic system 1 includes a first multicore optical fiber 11 and a second multicore optical fiber 12. The device 15 for acquiring and processing images includes:
an illumination source 16 configured to deliver a light beam to the first multicore optical fiber 11,
a first imaging sensor 18 configured to receive a light beam coming from the proximal end of the first multicore optical fiber 11,
a second imaging sensor 19 configured to receive a light beam coming from the proximal end of the second multicore optical fiber 12.

The device 15 for acquiring and processing images includes an optical separating system 22 disposed on the optical path between the proximal end of the multicore optical fiber 11 and the imaging sensor 18 and reflecting, in the direction of the proximal end of the multicore optical fiber 11, the light beam coming from the illumination source 16.

This example makes it possible to achieve a super-resolution insofar as it is possible to acquire two images on two imaging sensors. It is also possible to acquire the images one after the other with different wavelengths.

According to a fourth exemplary embodiment illustrated on FIG. 8, the endoscopic system 1 includes a first multicore optical fiber 11 and a second multicore optical fiber 12. The device 15 for acquiring and processing images includes:
a first illumination source 16 configured to deliver a light beam to the first multicore optical fiber 11,
a second illumination source 17 configured to deliver a light beam to the second multicore optical fiber 12,
a first imaging sensor 18 configured to receive a light beam coming from the proximal end of the first multicore optical fiber 11,
a second imaging sensor 19 configured to receive a light beam coming from the proximal end of the second multicore optical fiber 12.

The device 15 for acquiring and processing images includes an optical separating system 22 disposed on the optical path between the proximal end of each multicore optical fiber 11, 12 and the imaging sensor 18, 19 and reflecting, in the direction of the proximal end of each multicore optical fiber 11, 12, the light beam coming from the illumination sources 16, 17.

According to this example, it is possible to obtain images with twice as much resolution. One advantage of this solution is to be able to view tumors.

Note that on FIGS. 5 to 8, the proximal end of the multicore optical fibers, provided with the optical connector 13, is schematically represented as being directly connected to the device 15 for acquiring and processing images. Of course, the proximal end of the multicore optical fibers 11, 12, provided with the optical connector 13, can be located at the proximal part 2a of the insertion instrument such that an optical cable 21 provides the optical link between the optical connector 13 attached to the control handle 4 and the device 15 for acquiring and processing images.

In the same way, it must be considered that the device 15 for acquiring and processing images is configured in such a way as to convey the light between the imaging sensors 18, 19 and the optical connectors 13, 13a attached to the device 15 for acquiring and processing images. Similarly, the device 15 for acquiring and processing images is configured in such a way as to convey the light by any appropriate means between the illumination sources 16, 17 and the optical connectors 13, 13a attached to the device 15 for acquiring and processing images.

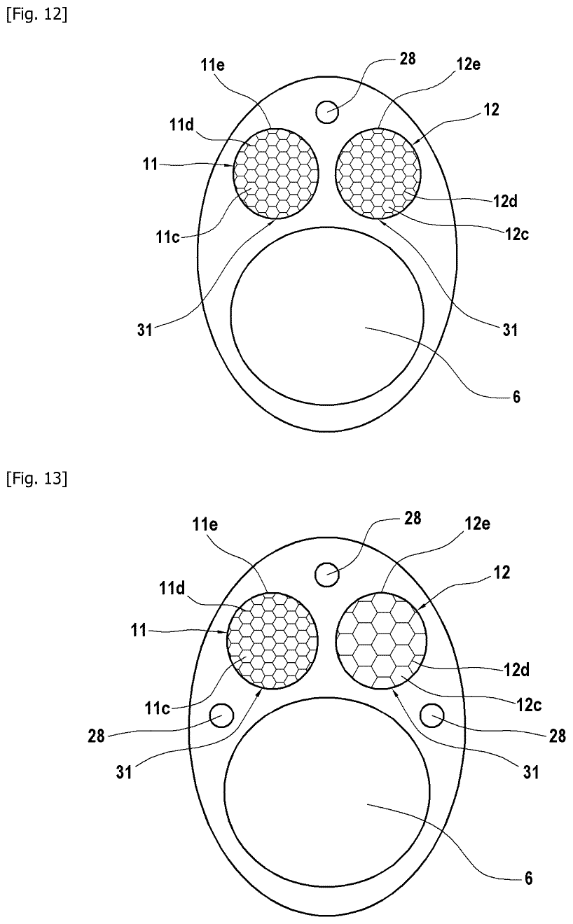

Note that according to FIGS. 5 to 8, the multicore optical fibers 11, 12 in particular convey the luminous flux from the illumination sources all the way to the distal head 2b of the insertion instrument. Note, as illustrated on FIGS. 11 and 12, that it may be envisioned for the medical endoscopic system 1 to include at least one illuminating optical fiber 28 and in the example illustrated in FIG. 13, three illuminating optical fibers allowing the delivery of an additional luminous flux. This illuminating optical fiber 28 has a distal end 28a and a proximal end 28b collecting the luminous flux from a light source 29. The distal end 28a of the illuminating optical fiber 28 is located at the distal head 2b of the insertion instrument while the proximal end of the illuminating optical fiber is located at the proximal part 2a of the insertion instrument while being provided with an optical connector through which is conveyed an illuminating light beam supplied by the light source 29.

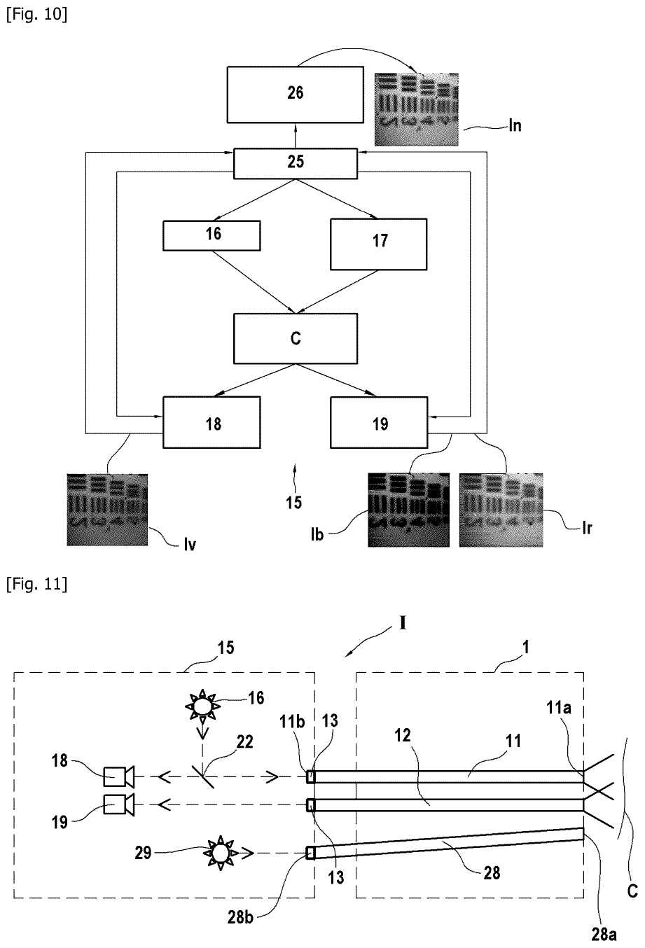

The device 15 for acquiring and processing images also includes, as illustrated on FIG. 10, an imaging processor 25 linked to the imaging sensors 18, 19 and configured to form images of the target C, based on the signals delivered by the imaging sensors 18, 19. The imaging processor 25 controls the imaging sensors 18, 19 to acquire the images of the target at given times. The imaging processor 25 also controls the illumination sources 16, 17 to control the illumination emitted, particularly during the acquisition of the images by the imaging sensors 18, 19 as described in the remainder of the description. The imaging processor 25 is connected to a viewing screen 26 used to display the images of the target C. This viewing screen 26 can be part of the device 15 for acquiring and processing images or be remote from this device. Of course, the imaging processor 25 can be connected to a memory to record the images.

The device 15 for acquiring and processing images can take different forms. Conventionally, the device 15 for acquiring and processing images can take the form of an electronic tablet provided with the viewing screen 26 and with a human-machine interface allowing a user to enter data or to control this device. This human-machine interface can be a keyboard, a mouse, or the screen for example embodied by a touch-sensitive screen. The device 15 for acquiring and processing images also includes a communication unit configured to communicate with a database, generally remote, forming part of a computer system.

The imaging system I according to the invention can be implemented in different ways which arise directly from the preceding description.

According to an exemplary implementation, the illumination source or sources 16, 17 are configured to deliver light beams in different wavelength spectra and the imaging sensor or sensors 18, 19 are suitable for acquiring images of different wavelength spectra. Typically, it may be envisioned to acquire images with different acquisition times before reconstructing them. Advantageously, the imaging processor 25 processes the images of different wavelength spectra to obtain a spectral super-resolution image. In other words, the resultant image has a greater resolution than the resolution of the images taken.

According to an advantageous exemplary embodiment, the illumination source or sources 16, 17 are configured to deliver light beams in the red, green and blue wavelength spectra and the imaging sensor or sensors 18, 19 are configured to acquire images in the red Ir, green Iv and blue Ib wavelength spectra. In the example illustrated on FIG. 10, the first illumination source 16 is controlled to deliver a light beam in a red wavelength spectrum and the first imaging sensor 18 is configured to acquire images in the red Ir wavelength spectrum. The second illumination source 17 is configured to successively deliver light beams in green and blue wavelength spectra and the second imaging sensor 19 is configured to acquire images in the green Iv and blue Ib wavelength spectra.

Moreover, the imaging processor 25 processes the wavelength spectrum images to obtain a contrasted or colored image In which can be a white image.

In the illustrated example, the imaging processor 25 processes the images in the red Ir, green Iv and blue Ib wavelength spectra to obtain a white image In. Typically, for an imaging sensor 18, 19 of CMOS type with a BAYER matrix, each red Ir, green Iv and blue Ib wavelength spectrum for example has a resolution of 40 000 pixels. The inclusion of these images makes it possible to obtain a white image of a resolution of 120 000 pixels.

According to another advantageous exemplary embodiment, the illumination source or sources 16, 17 are configured to successively deliver light beams in different wavelength spectra such infrared light radiation and ultraviolet light radiation.

According to another exemplary implementation, the imaging processor 25 controls the imaging sensor or sensors 18, 19 to acquire temporally-offset images. The imaging processor 25 processes the temporally-offset images to obtain a temporal super-resolution image. Thus, the imaging processor 25 processes a series of images taken successively over time in such a way as to obtain a resultant image with an improved resolution by comparison with the resolution of each image taken.

According to another exemplary implementation, the imaging processor 25 controls the imaging sensor or sensors 18, 19 to acquire images that are spatially offset while having an area of overlap. These images are spatially offset following the displacement of the insertion instrument 2 or given the offset of the two multicore optical fibers at the distal head 2b. The imaging processor 25 processes the spatially but also temporally offset images to obtain a spatial super-resolution image. Thus, the imaging processor 25 processes a series of images taken successively for different positions of the distal head in such a way as to obtain a resultant image with an improved resolution by comparison with the resolution of each image taken.

The spatial, temporal and spectral super-resolution images are produced using image processing algorithms based on multi-image super-resolution methods. These methods are based on three different known approaches: Interpolation Based approaches; Frequency domain-based approaches; Reconstruction based approaches. These methods are briefly described in particular in the following publications: 1—S. Borman and R. Stevenson, Super-Resolution from Image Sequences: A Review, in Midwest Symposium on Circuits and Systems, Notre Dame, IN, USA, 8 1998, pp. 374-378. S. C. Park, M. K. Park, and M. G. Kang. 2—Super-Resolution Image Reconstruction: A Technical Overview, IEEE Signal Processing Magazine, vol. 20, no. 3, pp. 21-36, 5 200. 3—C. Mancas-Thillou and M. Mirmehdi, An Introduction to Super-Resolution Text, in Digital Document Processing, ser. Advances in Pattern Recognition. Springer London, 2007, pp. 305-327. 4—Tian and K. -K. Ma, A survey on super-resolution imaging, Signal, Image and Video Processing (SIVIP), vol. 5, no. 3, pp. 329-342, 2011.

As a result of the various preceding variant embodiments, at least one imaging sensor 18, 19 is configured to receive light beams coming from the proximal end of the first multicore optical fiber 11 and from the proximal end of the second multicore optical fiber 12. In accordance with the invention, the imaging sensor 18, 19 receives the images from a configuration system 31 to create, on the imaging sensor 18, 19, images of the target C, for which the position, with respect to the target, of the separating structure or matrix 11d of the first multicore optical fiber 11 is different from the position, with respect to the target, of the separating structure or matrix 12d of the second multicore optical fiber 12.

The imaging processor 25 is configured to process the images coming from the first multicore optical fiber 11 and from the second multicore optical fiber 12, to reconstitute a composite image of the target in which the image of the matrices or of the separating structures 11d, 12d does not appear. Specifically, given the presence of a separating structure 11d, 12d separating the cores 11c, 12c for each multicore optical fiber 11, 12, the images I11 and I12 (FIG. 4) taken based on the luminous radiation respectively conveyed by the first multicore optical fiber 11 and the second multicore optical fiber 12, make dark areas G in the shape of grids or honeycombs appear, corresponding to the images of the separating structure 11d, 12d of the multicore optical fibers. These dark areas G correspond to areas of the target C which are not observed by the imaging sensors.

The objective of the principle of the invention is for the luminous fluxes conveyed by the first multicore optical fiber 11 and the second multicore optical fiber 12 to cover an entire area of the target C. Insofar as the position, on the imaging sensor, of the matrix 11d of the first multicore optical fiber 11 is different from the position, on the imaging sensor, of the matrix 12d of the second multicore optical fiber 12, the entire area of the target C is then observed. Of course, the observed area of the target C corresponds to the shared area of the target observed by both the multicore optical fibers 11, 12. Note that the fields of view of the two multicore optical fibers may not coincide. In this case, only the shared part of the fields of view of the two multicore optical fibers allow the reconstruction of a composite image devoid of the image of the separating structures 11d, 12d.

The imaging processor 25 is configured to construct a composite image Ic based on the images I11 coming from the first multicore optical fiber 11 and on the images I12 coming from the second multicore optical fiber 12. The missing parts of an image coming from the first multicore optical fiber 11 and corresponding to the dark area G are reconstituted based on the parts of an image coming from the second multicore optical fiber 11 and not corresponding to a dark area of said image. In other words, the missing part of an image coming from the first multicore optical fiber is completed by the corresponding part of an image coming from the second multicore optical fiber. Of course, conversely, the missing part of an image coming from the second multicore optical fiber is completed by the corresponding part of an image coming from the first multicore optical fiber. In the composite image Ic thus constructed, the image of the matrices, i.e. the image of the separating structure 11d, 12d of the cores of the two multicore optical fibers 11, 12, as illustrated on FIG. 4, does not appear. Such a super-resolved image Ic makes it possible to obtain an image of the entirety of the target with improved resolution. Within the meaning of the invention, it is permissible for the composite image Ic thus reconstructed to include one or more indefinite areas corresponding to dark areas.

Of course, the imaging processor 25 implements image-processing programs making it possible, based on the images I11 and I12 taken from the multicore optical fibers 11, 12, to construct a composite image. Such image-processing programs can use neural networks for which a training phase has been executed with target reference images.

Of course, the configuration system 31 for creating images of the target C, for which the position, on the imaging sensor, of the matrix 11d of the first multicore optical fiber 11 is different from the position, on the imaging sensor, of the matrix 12d of the second multicore optical fiber 12 can be embodied in any appropriate way.

According to an advantageous feature of the invention, at least one variant embodiment of the configuration system 31 described hereinafter is suitable for creating, on the imaging sensor or sensors, images coming from the two multicore optical fibers and containing size indicators of the target. The images thus created are processed by the imaging processor 25 to determine a measurement of the target based on the size indicators present on the images. As can be seen from the examples described hereinafter, the target size indicators are related to the physical features of the multicore optical fibers 11, 12 such as for example the diameter of the cores 11c, 12c, the diameter of these multicore optical fibers, the thickness of the matrices 11d, 12d or the shapes of the sections of the multicore optical fibers 11, 12.

According to an advantageous variant embodiment, the configuration system 31 is embodied by the first multicore optical fiber 11 and the second multicore optical fiber 12 having different orientations (FIG. 12). According to this example, the first multicore optical fiber 11 and the second multicore optical fiber 12 are of a same type of fiber and the distal ends 11a, 12a of these multicore optical fibers 11, 12 are positioned at the distal head 2b in such a way that these multicore optical fibers 11, 12 observe the target with separating structures 11d, 12d spatially offset from one another. Note that two multicore optical fibers 11, 12 can be used to take measurements of the dimensions of the target based on the images of the target taken by these two multicore optical fibers. Specifically, the separating structures 11d, 12d may serve as size indicators to determine a size measurement of the target.

According to another advantageous variant embodiment, the configuration system 31 is embodied by the first multicore optical fiber 11 having cores 11c of a given diameter while the second multicore optical fiber 12 has cores 12c with a different diameter from the diameter of the cores of the first optical fiber. In the example illustrated in FIG. 13, the cores 11c of the first multicore optical fiber 11 have, by way of example, a smaller diameter than the cores 12c of the second multicore optical fiber 12.

According to the example above, the two multicore optical fibers 11, 12 have the same section i.e. the same diameter. Note that according to another advantageous variant embodiment, the configuration system 31 can be embodied by the first multicore optical fiber 11 and the second multicore optical fiber 12 which have different sections. Thus, the diameter of the first multicore optical fiber 11 can be less than the diameter of the second multicore optical fiber 12. Note that the implementation of two multicore optical fibers 11, 12 of different dimensions can be used as size indicators to take dimension measurements of the target based on the images of the target taken by these two multicore optical fibers.

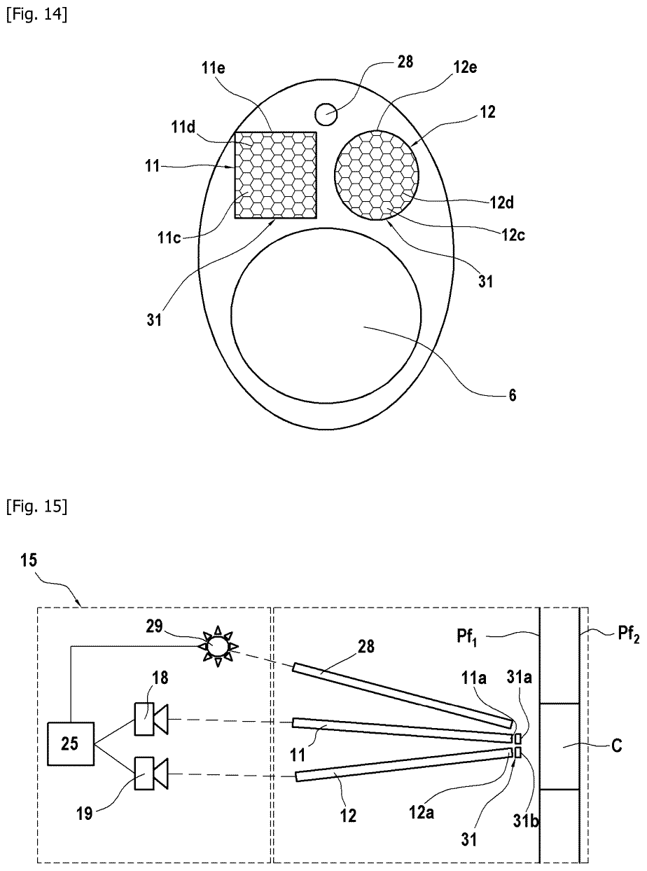

According to another advantageous variant embodiment, the configuration system 31 is embodied by the first multicore optical fiber 11 having a section of given shape while the second multicore optical fiber 12 has a section of different shape to the shape of the section of the first optical fiber. In the example illustrated in FIG. 14, the first multicore optical fiber 11 has a square section while the second multicore optical fiber 12 has a round section. Note that the implementation of two multicore optical fibers 11, 12 with sections of different shapes can be used as size indicators to take dimension measurements of the target based on the images of the target taken by these two multicore optical fibers. Specifically, the dimensions of the sections of these two multicore optical fibers being known, it is thus possible to take measurements of the target on the images taken by these two multicore optical fibers.

According to another advantageous variant embodiment, the configuration system 31 is embodied by an optical system 31a, 31b disposed at the distal end of the multicore optical fibers 11, 12 to create different field depths. As can be seen from FIG. 15, the optical system can for example include a first lens 31a disposed at the distal end 11a of the first multicore optical fiber 11 to observe the target C at a field depth Pf1. A second lens 31b is disposed at the distal end 12a of the second multicore optical fiber 12 to observe the target C at a field depth Pf2 which is different from the field depth Pf1.

According to another advantageous variant embodiment, the configuration system 31 is embodied by the use of a first multicore optical fiber 11 and of a second multicore optical fiber 12 having different numerical apertures. Thus, as the aperture of a multicore optical fiber 11, 12 defines its field of view, the images obtained by multicore optical fibers of different apertures are different.

It can be seen from the preceding description that the configuration system 31 can be embodied by one and/or the other of the variant embodiments described above. In other words, it must be understood that these various variant embodiments of the configuration system 31 can be combined with one another in any appropriate way. For example, the multicore optical fibers 11, 12 may have cores of different diameters but also of different section shapes.

According to another advantageous variant embodiment, the configuration system 31 is configured so that the images I11 coming from the first multicore optical fiber 11 and the images 112 coming from the second multicore optical fiber 12, can be matched. Specifically, to reconstruct the missing parts of an image via the corresponding parts of another image, it is advisable for the positions of the images to be referenced with one another. The determination of the relative positions of the images with respect to one another can be done in any appropriate way. For example, the implementation of a reference frame or a mark appearing on the images taken allows this matching insofar as the relative positioning of the distal ends 11a, 12a of the multicore optical fibers 11, 12 at the distal head 2b of the insertion instrument is known.

Note that the implementation of two multicore optical fibers 11, 12 with different section shapes can be used to match the images taken by the multicore optical fibers 11, 12. Specifically, the relative positioning of the multicore optical fibers 11, 12 at the distal head 2b of the insertion instrument being known, it is possible to determine the relative position of the images based on their characteristic shape. Advantageously, the imaging processor 25 processes the images coming from the two multicore optical fibers 11, 12 to determine an orientation of the distal head 2b. Specifically, the images coming from the two multicore optical fibers 11, 12 with different section shapes have orientation indicators making it possible to determine the orientation of the distal head 2b since the relative orientation of the two multicore optical fibers 11, 12 is known at the distal head 2b.

According to another aspect, an imaging system I including two multicore optical fibers 11, 12 with sections of different shapes can be advantageously used to assist in the location of the distal head with respect to the target.

1. An imaging system for a medical endoscopic system for viewing a target including:
a medical endoscopic system including an insertion instrument terminating, opposite a proximal part, in a distal head, this insertion instrument being provided with a first multicore optical fiber and with a second first multicore optical fiber each having a distal end, a proximal end and a plurality of cores separated by a separating structure, the distal ends of the first multicore optical fiber and of the second multicore optical fiber being located at the distal head of the insertion instrument; and
a device for acquiring and processing images including:
at least a first illumination source configured to deliver a light beam, in at least a first wavelength spectrum, to the distal head of the insertion instrument;
at least one imaging sensor configured to receive light beams coming from at least the proximal end of the first multicore optical fiber and from the proximal end of the second multicore optical fiber, the imaging sensor receiving the images from a configuration system to create, on the imaging sensor, images of the target, for which the position of the separating structure of the first multicore optical fiber is different from the separating structure of the second multicore optical fiber;
an imaging processor connected to the imaging sensor and configured to process the images coming from the first multicore optical fiber and from the second multicore optical fiber, to reconstitute a composite image of the target in which the image of the separating structures does not appear.

2. The imaging system as claimed in claim 1, wherein the device for acquiring and processing images includes a single imaging sensor configured to either receive, on two separate areas, the light beams coming from the first multicore optical fiber and from the second multicore optical fiber, or to receive, on a shared but time-offset area, the light beams coming from the first multicore optical fiber and from the second multicore optical fiber.

3. The imaging system as claimed in claim 1, wherein the device for acquiring and processing images includes two imaging sensors configured to each receive a light beam coming either from the first multicore optical fiber or from the second multicore optical fiber.

4. The imaging system as claimed in claim 1, wherein the configuration system is configured so that the images coming from the first multicore optical fiber and the images coming from the second multicore optical fiber can be matched.

5. The imaging system as claimed in claim 1, wherein the imaging processor processes the images to determine an orientation of the distal head.

6. The imaging system as claimed in claim 1, wherein the configuration system is embodied by the first multicore optical fiber and the second multicore optical fiber having sections of different shapes.

7. The imaging system as claimed in claim 1, wherein the configuration system is embodied by the first multicore optical fiber and the second multicore optical fiber having different orientations.

8. The imaging system as claimed in claim 1, wherein the configuration system is embodied by a first multicore optical fiber and a second multicore optical fiber having different numerical apertures.

9. The imaging system as claimed in claim 1, wherein the configuration system is embodied by an optical system disposed at the distal end of the multicore optical fibers to create different field depths.

10. The imaging system as claimed in claim 1, wherein the configuration system is embodied by the first multicore optical fiber having cores of a given diameter while the second multicore optical fiber has cores with a different diameter from the diameter of the cores of the first optical fiber.

11. The imaging system as claimed in claim 1, wherein the configuration system is embodied by the first multicore optical fiber having a section of given shape while the second multicore optical fiber has a section of different shape from the shape of the section of the first multicore optical fiber.

12. The imaging system as claimed in claim 1, wherein the illumination source is configured to deliver a light beam to at least one multicore optical fiber.

13. The imaging system as claimed in claim 1 wherein an illumination source is configured to deliver a light beam to at least one optical fiber conveying the light all the way to the distal head of the insertion instrument.

14. The imaging system as claimed in claim 1, wherein the imaging sensor or sensors receive the images from the configuration system configured to create, on the imaging sensor or sensors, images coming from the two multicore optical fibers and containing size indicators of the target and in that the imaging processor processes the images to determine a measurement of the target based on the size indicators.
